(19) United States
(12) Patent Application Publication
(10) Pub. No.: US 2020/0383806 A1
(43) Pub. Date: Dec. 10, 2020

(54) LINER FOR A TRANSRADIAL PROSTHESIS AND METHOD FOR THE PRODUCTION THEREOF

(71) Applicant: Ottobock SE & Co. KGaA, Duderstadt (DE)

(72) Inventor: Mario KOPPE, Göttingen (DE)

(73) Assignee: Ottobock SE & Co. KGaA, Duderstadt (DE)

(21) Appl. No.: 15/733,068

(22) PCT Filed: Oct. 16, 2018
(86) PCT No.: PCT/EP2018/078234

§ 371 Date: May 6, 2020

(30) Foreign Application Priority Data
Nov. 10, 2017 (DE) 10 2017 126 472.8

(51) Int. Cl. A61F 2/78; A61F 2/72; A61F 2/50; A61F 2/80; A61B 5/00

(52) U.S. Cl. CPC A61F 2/7812; A61F 2/72; A61F 2/5046; A61F 2002/7837; A61B 5/6811; A61F 2002/5053; A61F 2/80

(57) ABSTRACT

A liner and a method for producing a liner for a lower arm prosthesis, wherein the liner comprises a base body with a closed distal end and an open proximal end, and the method comprises the production of the base body by moulding a preform, in particular using a casting or dipping method, wherein in at least one section of the preform a first diameter of the blank is greater than a second diameter in a second direction, which extends orthogonally to the first direction.

The invention relates to a liner for a prosthesis system, wherein the liner comprises a base body with a closed distal end, an open proximal end and a longitudinal extension that extends from the distal end to the proximal end.

Prosthesis liners for a range of different prostheses are known from the prior art. They are worn directly on the amputation stump. In general, they are rolled up prior to applying, then applied on the distal end of the amputation stump and unrolled over the amputation stump like a stocking. Due to the relatively tight fit and the liner material, which is preferably a silicone or polyurethane, a subsequent displacement of the liner on the amputation stump can only be achieved with great difficulty, or not at all.

Conventional prostheses often have an electric or electronic control system which can be used, for instance, to control functions of the prosthesis. The electrical signals required for this are often picked up by the amputation stump in the form of bio-signals. Bio-signals are, for example, myoelectric signals or bio-impedance signals. This renders it possible for the wearer of the prosthesis to control the prosthesis in an easy and intuitive manner. This is especially prevalent and advantageous in the case of lower arm and hand prostheses. Alternatively or additionally, it is also advantageous to introduce electrical signals into the amputation stump via an electric or electronic control system, for example in order to stimulate muscle groups on the amputation stump. Regardless of the direction in which electrical signals are to be picked up or emitted, for such applications it is necessary for the liner for the lower arm prosthesis, the base body of which is composed of a material that is not electrically conductive, to feature electrically conductive elements, for instance in the form of electrically conductive through-lines or electrodes that that integrated in the liner itself, in order to be able to conduct the electrical signals through the liner. To ensure that the right electrical signals can be transmitted to the body of the wearer through the through-lines and/or electrodes arranged on the liner, or can be picked up by the body at the right points, it must be ensured that the contact points enabled by the liner are reproducible. For the wearer of the prosthesis and the prosthesis liner, this poses a challenge as it is very difficult or in many cases impossible to displace or twist the liner after it has been applied, as previously described. Should it become evident that the liner has been arranged in a false orientation or position on the amputation stump and the electric through-lines and/or electrodes therefore do not lie at the desired points on the amputation stump, the liner must be removed from the amputation stump and re-applied in a new position and/or orientation. This is an inconvenient and potentially long procedure and leads to a low degree of acceptance and/or incorrect or poor electrical signals.

The invention thus aims to propose a liner for a prosthesis system, especially a lower arm prosthesis, a corresponding liner and a method for producing such a liner, which can be produced in a simple and cost-effective manner and which guarantees a high quality of the electrical signals to be transmitted.

The invention solves the problem by way of a liner according to the generic term in claim 1, characterized in that a first diameter of the base body is greater in a first direction than a second diameter in a second direction, which extends orthogonally to the first direction.

The first diameter is preferably greater than the second diameter across at least 50%, preferably at least 75%, especially preferably 90%, especially preferably 100% of the longitudinal extension. A ratio of the first diameter to the second diameter preferably varies.

Preferably, the liner features at least one electrical through-line and/or one electrode, which is arranged on or in the base body in such a way that, when the liner is applied, the through-line and/or the electrode comes into contact with a skin of the wearer. The through-lines may be designed to take different forms. For example, they may be moulded into the base body of the liner in the form of electrically conductive polymers, or produced when the liner is cast. To this end, it is common, for instance, to equip conventional electrically insulating polymers with additives which cause electrical conductivity. The at least one electrical through-line may, however, exist in the form of a through-line element made of a metal, for instance in the form of a button or a rivet. Of course, it is also possible to mould or glue a complete electrode into the liner material, or to provide a recess in the material of the base body into which an electrode is later inserted.

Preferably, the at least one through-line and/or the at least one electrode is connected to an electric conductor, which extends to an interface arranged on an outer side of the base body. The electrical signals that are to be transmitted via the through-line and/or the electrode to or from the skin of the wearer can be transported and conducted by way of the electric conductor. The conductor may be provided in the form of silver-coated fabric, for example, or in the form of moulded cables Preferably, cords or cables are used as electric conductors, wherein said cords or cables have been produced from an electrically conductive polymer, preferably the polymer from which the rest of the base body is also made. This ensures that the electric conductors have the same elasticity or at least a similar elasticity as the rest of the base body of the liner, so that application and removal of the liner, both of which subject the liner material to a considerable mechanical load, do not cause damage to the electric conductors.

In an especially preferred embodiment, the interface is arranged on the outer side in an extension of the second diameter. The second diameter is smaller than the first diameter, so that the liner has a smaller expansion in cross-section in this direction than in the first direction, which is perpendicular to the second. This preferably refers to a medial or lateral side of the liner. By arranging it at this point, the space required by the interface is arranged in a such a way that it causes as little disturbance as possible. Specifically, in this configuration the interface is not arranged at the distal end of the liner as is known from the prior art. Such an arrangement, which is of course also possible, has the disadvantage that the space required for the interface and the interface contacts to be provided in the prosthesis socket inevitably leads to an extension of the prosthesis structure. This is particularly disadvantageous in the case of lower arm prostheses, as the arm fitted with the prosthesis has an unnatural length, which is visually unattractive and noticeable, as well as being less comfortable to wear.

The invention also solves the problem by way of a prosthesis system, preferably a lower arm prosthesis with a prosthesis socket and a liner as described here, wherein the liner and the socket are designed in such a way that, when applied, an inner side of the prosthesis socket rests on an outer side of the liner.

The prosthesis socket preferably features at least one interface on an inner side, wherein said interface interacts with the interface arranged on the outer side of the liner in such a way that electrical signals can be conducted from and/or to the at least one through-line and/or electrode. It is especially advantageous if the outer side of the liner and the inner side of the prosthesis socket feature connection elements, preferably positive-locking elements, that correspond to one another, by way of which the liner can be connected to the prosthesis socket. On the one hand, this guarantees a secure connection between the liner and the prosthesis socket; on the other hand, it ensures that the electrical contacts can be established safely and easily. Due to its special form created by the method according to the invention, the liner must be arranged in such a way that it cannot be twisted, so that this alone ensures that the through-lines and/or electrodes are arranged at the correct point on the amputation stump and at the same time ensures that the interface for the electrical contact is also reproducibly arranged at the same point, preferably medially on the lower arm. In an especially preferred configuration, this results directly and automatically in an electrical contact between the interface on the inner side of the prosthesis socket and the interface on the outer side of the liner base body when the prosthesis socket is applied, and preferably also in a mechanical connection of the two components. This may result in a locking mechanism which, in addition to ensuring that the prosthesis socket is held securely on the prosthesis liner, also ensures that any tensile forces acting on the prosthesis socket are not transmitted via the electrical contact to the liner, but that the mechanical connection elements are used for this purpose.

The invention also solves the problem by way of a method for producing a liner for a lower arm prosthesis, wherein the liner comprises a base body with a closed distal end and an open proximal end, wherein the method comprises the production of the base body by moulding a preform, in particular using a casting or dipping method, wherein in at least one section of the preform a first diameter of the preform is greater than a second diameter in a second direction, which extends orthogonally to the first direction. A blank of a liner, which may have a circular cross-section in the region of the distal end, can also be placed on or pulled over a preform and subsequently shaped. Increased pressure and/or increased temperature, for example, may be used to achieve this.

Consequently, the liner to be produced, which gets its inner contour from the preform, does not have a circular inner cross-section in the at least one section which gets its shape from the at least one section of the preform. Rather, the diameter of the preform, which corresponds to the subsequent inner diameter of the liner to be produced, is greater in a first direction than in a second direction. Here, the two diameters lie in one plane and thus also in one cross-section. The cross-section is defined perpendicular to the longitudinal axis of the base body and thus perpendicular to the longitudinal axis of the preform. In the case of liners and thus also preforms which lend said liners their shape, it is common for these to feature a kink or a bend in order to render them more comfortable to wear on joints, especially knees and elbows. The longitudinal direction, perpendicular to which the cross-sections are defined in which the diameters are determined, extends from the distal closed end of the liner to the middle of the liner at the point that lies immediately before the kink. The kink or the bend itself does not influence the longitudinal direction described here.

For the sake of simplicity, the part of the preform which lends its shape to the end of the base body is described as the distal end of the preform and the part of the preform which lends its shape to the proximal end of the base body as the proximal end, even though such a description is of course purely arbitrary. The preform itself is not arranged on a body of a wearer, such that the immediate meaning of the words “distal” and “proximal” does not apply in this case.

The different sizes of the two diameters of the preform and therefore also the different sizes of the two inner diameters of the base body of the liner ensure that the liner can be arranged so that it cannot be twisted. Unlike with liners with a circular cross-section, the wearer notices very quickly whether the liner has been applied in an optimal position on the amputation stump.

The preform is preferably a convex preform. This means that an imaginary line between two arbitrary points on the surface of the preform is situated entirely within the preform. Specifically, a base body of a liner that has been produced with such a convex preform does not have any undercuts.

In a preferred configuration, there is no curvature in the wall of the base body of the liner along the longitudinal direction, i.e. moving away from the distal end, as soon as the distal end region, in which the wall must of course be curved, is completed. This does not mean that the size of the cross-section of the liner is constant. It is possible that the liner and also the preform which lends its shape to the liner expands towards the proximal end, so that the inner cross-section of the liner also increases. In this case, it is advantageous for the ratio of the first diameter to the second diameter to be constant within the at least one section of the preform. Cross-sections of the preform and therefore also of the liner in different planes may differ in this case in size, i.e. a scale, but not in the geometric form exhibited by the cross-section of the preform and the inner cross-section of the base body of the liner. In a preferred configuration, such a cross-section features at least one, but preferably two, axes of symmetry. For example, it may be an oval, an ellipsoid, a polygon or a rounded polygon. Of course, other cross-section shapes are also possible.

In a preferred configuration of the method, the ratio of the first diameter to the second diameter varies in the at least one section of the preform. This renders it possible to replicate, for instance, a shape of the lower arm stump in the case of lower arm prostheses, as the cross-section of a lower arm is approximately circular towards the elbow, whereas the diameter is very different towards a wrist.

Preferably, a diameter of the preform in at least one second section of the preform, which lies outside of the first section, is at least almost the same size, preferably exactly the same size, in all directions. This means that the preform in this second section has a circular cross-section.

In the following, examples of embodiments of the present invention will be explained in more detail by way of the figures: They show:

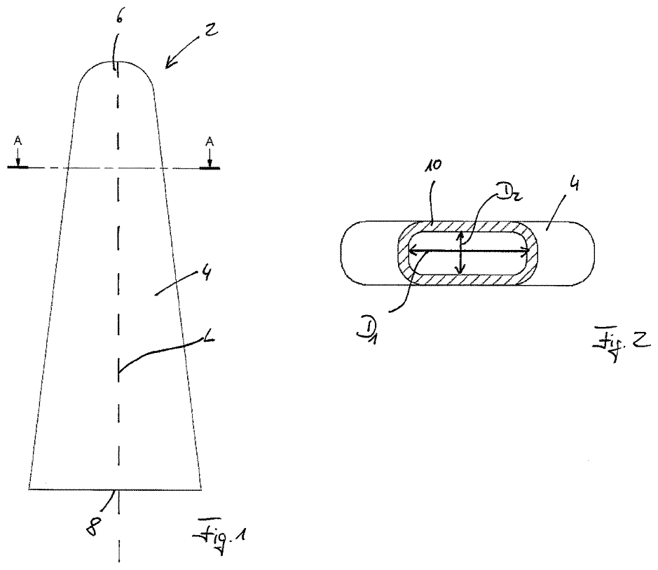

FIG. 1 the schematic side view of a liner according to a first example of an embodiment of the present invention;

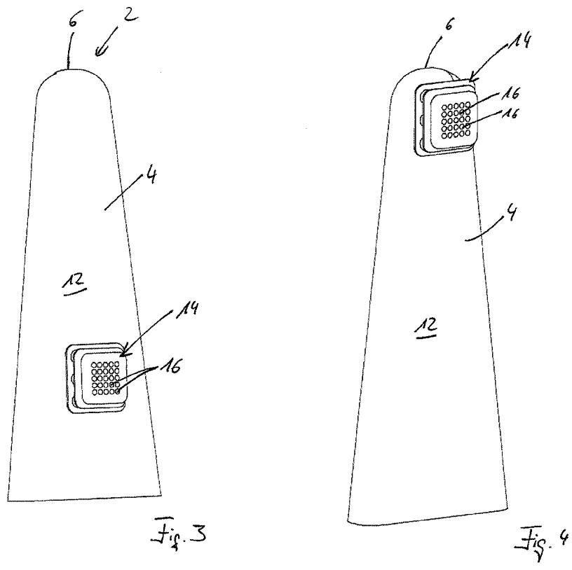

FIG. 2 a sectional view through the liner from FIG. 1 along the line A-A,

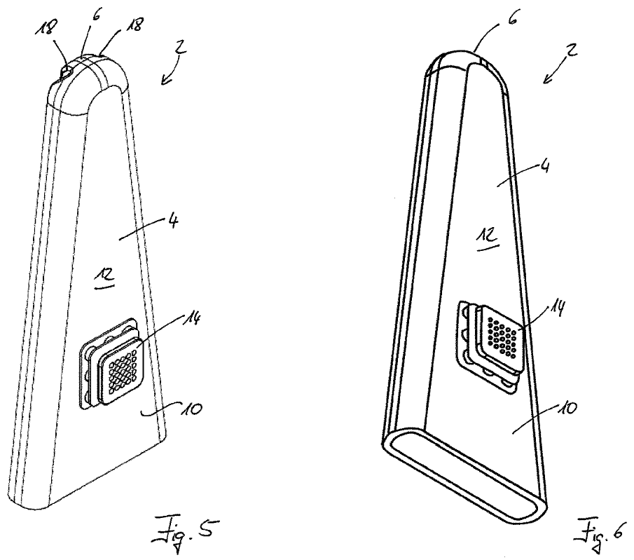

FIG. 3 the schematic representation of a liner according to a further example of an embodiment of the present invention;

FIG. 4 a further schematic representation of a liner according to an example of an embodiment of the present invention,

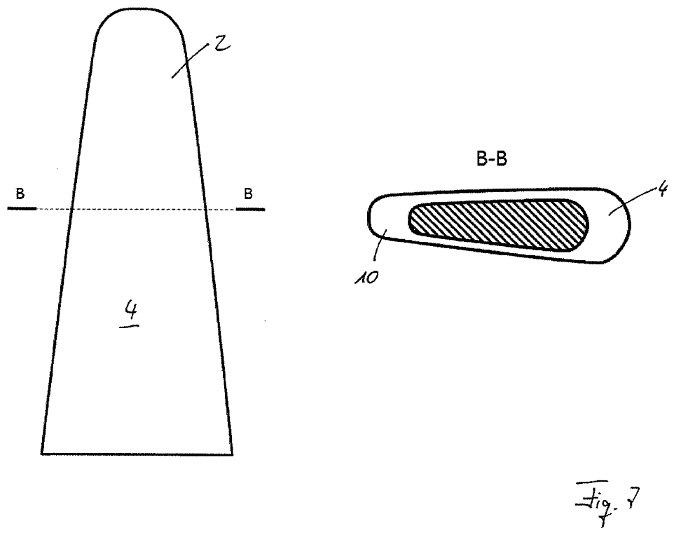

FIG. 5 the schematic three-dimensional view of a liner according to a further example of an embodiment of the present invention,

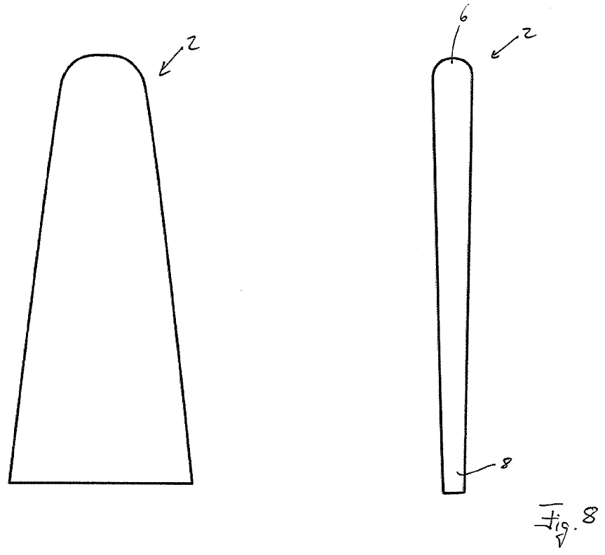

FIG. 6 the liner from FIG. 5 from another perspective,

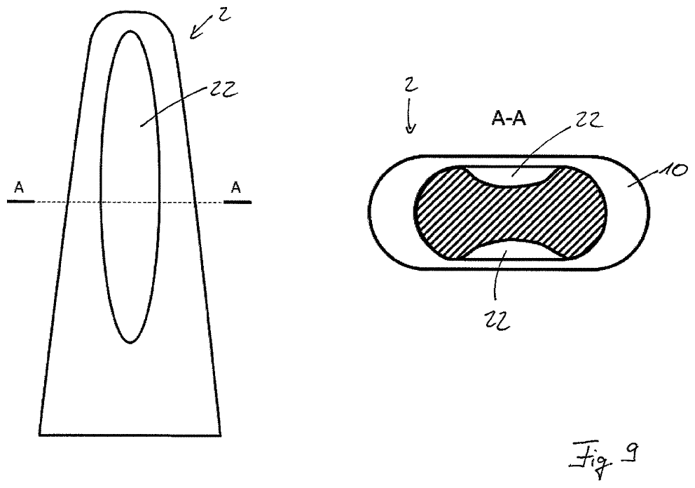

FIG. 7 a schematic side and sectional representation of a further embodiment of the liner,

FIG. 8 two schematic side views of a further embodiment and

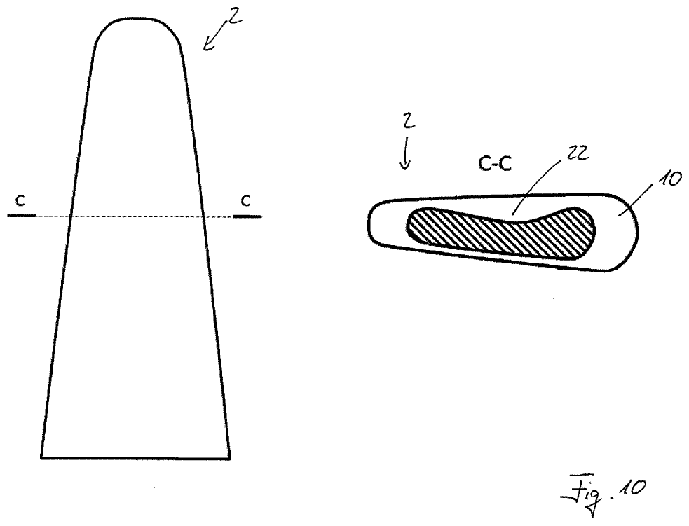

FIGS. 9 to 11—a schematic side and sectional representation of further embodiments of a liner according to the present invention.

FIG. 1 depicts a schematic side view of a liner 2 according to a first example of an embodiment of the present invention. A base body 4 of the liner comprises a closed distal end 6 and an open proximal end 8. It is clear that the base body 4 of the liner 2 expands along its longitudinal axis L towards the proximal end 8, so that a diameter increases in this direction.

FIG. 2 shows a cross section along the line A-A from FIG. 1. The base body 4, the expansion of which can be seen in the right and left-hand areas of FIG. 2, has a first diameter D₁ along a first direction, which extends from left to right in FIG. 2. Here, the first diameter D₁ is an inner diameter, which corresponds at least generally, but preferably precisely, to an outer diameter of a preform used during the production of the liner base body 4. A second diameter D₂, which is smaller than the first diameter D₁ as shown in FIG. 2, extends in a second direction, which extends perpendicular to the first direction. In the cross-section representation in FIG. 2, it can also be recognised that an expansion of the base body 4 of the liner only occurs along the first direction, along which the first diameter D₁ extends, but not along the second direction, along which the second diameter D₂ extends.

The base body 4 of the liner 2 depicted in FIG. 2 comprises a wall 10, which features a constant wall thickness across the entire circumference. This is an advantage but not absolutely necessary.

In the example of an embodiment shown in FIGS. 1 and 2, the diameter D₁ increases the further one moves along the longitudinal axis L away from the distal end 6. At the same time, the second diameter D₂ remains constant, so that the ratio of the two D₁:D₂ increases.

FIG. 3 shows a base body 4 of the liner 2, on the outer side 12 of which an interface 14 is arranged. It has a number of contacts 16 that interact with electrical through-lines and/or electrodes, which are arranged on the base body 4 but are not depicted in the figures. If the liner shown in FIG. 3 is now pulled over an amputation stump and then inserted into a prosthesis socket, said prosthesis socket preferably features a corresponding interface on its inner side which enables an electrical contact of the individual contacts 16. It is clear that the interface 14 is arranged on the outer side 12 of the base body 4 of the liner 2, not in the region of the distal end 6, but on a lateral surface of the liner.

A similar arrangement is shown in FIG. 4. Here, the interface 14 is also arranged with the contacts 16 on the outer side 12 of the base body 4; however, it is considerably closer to the distal end 6 than the case illustrated in FIG. 3. Nevertheless, the interface in both FIG. 3 and FIG. 4 is arranged on the outer side 12 of the base body 4 in an extension of the second diameter D₂, as shown in FIG. 2.

FIGS. 5 and 6 show a further example of an embodiment of the liner 2 according to the present invention. The base body 4 features an outer side 12, on which the interface 14 is arranged. In the example of an embodiment shown, the wall 10 in this region is straight, i.e. it is designed to be level and does not have any curvature. This is an advantage for some applications but not absolutely necessary. Mechanical connection elements 18 are depicted in the region of the distal end 6 of the liner 2, wherein said elements enable a connection of the liner 2 to the prosthesis socket, not depicted.

FIG. 7 shows the liner 2 with the base body 4 with the exterior structure previously shown in FIG. 1. The right-hand part depicts a sectional representation along the line B-B. The inner space is depicted by crosshatch, while the wall 10 is depicted with no crosshatch. The liner has a conical cross-section, so that an extension in FIG. 7 from top to bottom is smaller in the left-hand area than in the right-hand area. This renders it possible to respond to certain requirements, especially regarding the anatomy of the limb being treated.

FIG. 8 depicts two exterior representations of a liner from different perspectives. While the representation in the left-hand area of FIG. 8 corresponds to the left-hand representation in FIG. 7 as well as the representation in FIG. 1, it is clear in the right-hand view that a thickness of the liner, i.e. the extension from left to right in FIG. 8, reduces from the distal end 6 towards the proximal end 8 and the liner tapers.

The left-hand side of FIG. 9 depicts the liner 2 in a representation from the outside. It contains a schematic representation of a thickening 22, which can be seen along the line A-A in the sectional representation shown on the right. The inner space of the liner 2 features two thickenings 22, which are arranged as separate elements on the inner side of the wall 10 in the example of an embodiment shown. Of course, they may also be designed as a single piece with the wall 10. This renders it possible to take anatomical circumstances into account and to individually adjust the liner 2 to the required fit. In this case, it is not necessary for the two thickenings 22 to be designed to be identical. In certain cases, it may be advantageous to provide just one thickening 22 or to arrange differently designed thickenings 22 on the inner side of the wall 10 or to design them as single pieces with said wall.

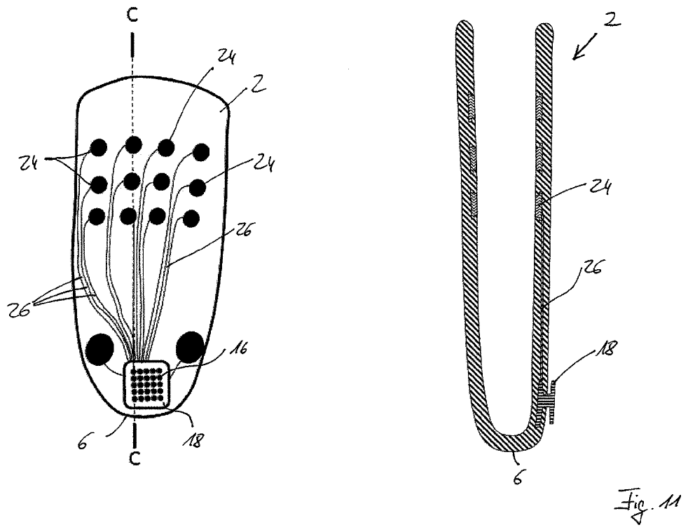

FIG. 10 shows a combination of the described properties. The exterior representation of the liner 2 in the currently known form is once again depicted in the left-hand area. To the right in the sectional representation, along the line C-C, it can be seen that the outer contour of the liner 2 in FIG. 10 tapers from right to left and the cross-section is designed to be conical. At the same time, the inner side of the wall 10 features a thickening 22, which is designed as a single piece with the wall 10 and is also designed to be asymmetrical. Only one thickening 22 is provided, so that the opposite side of the wall does not have a thickening.

The left-hand representation in FIG. 11 depicts a liner 2 for an example of an embodiment of the present invention. It features several electrodes 24, each of which is connected to the electrical contacts 16 via an electric conductor 26. The electric conductors 26 extend inside the material of the liner 2. The right-hand representation in FIG. 11 depicts the sectional view along the line C-C. The liner 2 with the electric conductors 26 inside the liner material can be clearly seen, wherein said electric conductors lead to the electrodes 24.

The contacts 16 form part of a connection element 18, by means of which the liner 2 can be connected to a prosthesis socket, not depicted; in particular, said connection element enables a mechanical locking of the liner.

### REFERENCE LIST

L longitudinal axis
D₁ first diameter
D₂ second diameter
2 liner
4 base body
6 distal end
8 proximal end
10 wall
12 outer side
14 interface
16 contact
18 connection element
22 thickening
24 electrode
26 electric conductor

1. A liner for a prosthesis system, the liner comprising:
a base body having a closed distal end, an open proximal end, and a longitudinal extension that extends from the distal end to the proximal end;
wherein a first diameter of the base body at the distal end is greater in a first direction than a second diameter in a second direction, which extends orthogonally to the first direction.

2. The liner according to claim 1, wherein the first diameter is greater than the second diameter across at least 50% of the longitudinal extension.

3. The liner according to claim 2, wherein a ratio of the first diameter to the second diameter varies.

4. The liner according to claim 1, wherein the liner includes at least one of an electrical through-line and an electrode, which is arranged on or in the base body in such a way that, when the liner is applied, the at least one of the through-line and the electrode comes into contact with a skin of the wearer.

5. The liner according to claim 4, wherein the at least one of the through-line and the electrode is connected to an electric conductor, which extends to an interface arranged on an outer side of the base body.

6. The liner according to claim 5, wherein the interface is arranged on the outer side in an extension of the second diameter.

7. A lower arm prosthesis with a prosthesis socket and a liner according to claim 1, wherein the liner and the prosthesis socket are designed in such a way that, when applied, an inner side of the prosthesis socket rests on an outer side of the liner.

8. The lower arm prosthesis according to claim 7, wherein the prosthesis socket includes at least one interface on an inner side that interacts with an interface arranged on the outer side of the liner in such a way that electrical signals can be conducted from or to at least one of a through-line and an electrode.

9. The lower arm prosthesis according to claim 7, wherein the outer side of the liner and inner side of the prosthesis socket feature connection elements that correspond to one another, by way of which the liner can be connected to the prosthesis socket.

10. A method for producing a liner having the features of claim 1, wherein the method comprises:
producing the base body by moulding a preform, wherein in at least one first section of the preform a first diameter of the preform in a first direction is greater than a second diameter in a second direction, which extends orthogonally to the first direction of the preform.

11. The method according to claim 10, wherein a diameter of the preform in at least one second section of the preform, which lies outside of the first section, is at least almost the same size in all directions.

12. A prosthesis liner comprising:
a base body comprising:
a closed distal end;
an open proximal end;
a longitudinal extension extending from the distal end to the proximal end;
a first diameter measured in a first direction at the distal end;
a second diameter measured in a second direction at the distal end, the second direction being orthogonal to the first direction.

13. The liner according to claim 12, wherein the first diameter is greater than the second diameter across at least 50% of the longitudinal extension.

14. The liner according to claim 12, wherein a ratio of the first diameter to the second diameter is variable.

15. The liner according to claim 12, wherein the liner includes at least one of an electrical through-line and an electrode, which is arranged on or in the base body in such a way that, when the liner is applied, the at least one of the through-line and the electrode comes into contact with a skin of the wearer.

16. The liner according to claim 12, wherein the at least one of the through-line and the electrode is connected to an electric conductor, which extends to an interface arranged on an outer side of the base body.

17. The liner according to claim 12, wherein the interface is arranged on the outer side in an extension of the second diameter.

18. The liner according to claim 12, wherein the first diameter is greater than the second diameter across 100% of the longitudinal extension.

19. A method for producing a prosthetic liner, comprising:
moulding a base body from a preform, the preform having a closed distal end, an open proximal end, a longitudinal extension extending from the distal end to the proximal end, and a first section, the first section having a first diameter measured in a first direction at the distal end, and a second diameter measured in a second direction at the distal end, the second direction being orthogonal to the first direction.

20. The method according to claim 19, wherein a diameter of the preform in at least one second section of the preform, which lies outside of the first section, is the same size in all directions.
